(19) United States
(12) Patent Application Publication
(10) Pub. No.: US 2025/0195805 A1
(43) Pub. Date: Jun. 19, 2025

(54) System and Methods of Administering a Status Check to a Medical Device

(71) Applicant: Ventis Medical, Inc., Princeton, NJ (US)

(72) Inventors: Glenn W. LAUB, Princeton, NJ (US); Giovanni MEIER, Madison, CT (US); Bob GORRY, New Haven, CT (US); David G. LAUB, Princeton, NJ (US); Taylor R. LAUB, Princeton, NJ (US); Steven JACOBSON, New York, NY (US); Karen LAUB, Princeton, NJ (US)

(21) Appl. No.: 18/846,896

(22) PCT Filed: Mar. 15, 2023
(86) PCT No.: PCT/US2023/064409

§ 371 Date: Sep. 13, 2024

Related U.S. Application Data
(60) Provisional application No. 63/320,640, filed on Mar. 16, 2022.

(51) Int. Cl. A61M 16/00

(52) U.S. Cl. CPC A61M 16/022; A61M 16/0051; A61M 2205/18; A61M 2205/3306; A61M 2205/3368; A61M 2205/502; A61M 2205/581; A61M 2205/702

(57) ABSTRACT

A medical device includes an electro-mechanical pneumatic system disposed within a housing of the medical device and coupled to one or more components, and a control system coupled to the electro-mechanical pneumatic system. The control system is configured to without receiving an external request, automatically transmit to one or more of the electro-mechanical pneumatic system and the one or more components a request for status data regarding a status of one or more of the electro-mechanical pneumatic system and the one or more components, receive the status data from one or more of the electro-mechanical pneumatic system and the one or more components, and output a signal based on the status data from one or more of the electro-mechanical pneumatic system and the one or more components.

### CROSS-REFERENCE TO RELATED APPLICATIONS

This application claims the benefit of U.S. Provisional Patent Application No. 63/320,640 filed Mar. 16, 2022 entitled “System and Methods of Administering a Status Check to a Medical Device”, which is incorporated by reference herein in its entirety.

### TECHNICAL FIELD

The present disclosure generally relates to a system and method of administering a status check to a medical device and, in some embodiments, to system and method for administering a status check to a ventilator.

### SUMMARY

Embodiments of the present invention are directed to a medical device including an electro-mechanical pneumatic system disposed within a housing of the medical device and coupled to one or more components, and a control system coupled to the electro-mechanical pneumatic system, the control system configured to without receiving an external request, automatically transmit to one or more of the electro-mechanical pneumatic system and the one or more components a request for status data regarding a status of one or more of the electro-mechanical pneumatic system and the one or more components, receive the status data from one or more of the electro-mechanical pneumatic system and the one or more components, and output a signal based on the status data from one or more of the electro-mechanical pneumatic system and the one or more components.

In some embodiments, the medical device further includes a transmitting device configured to receive the status data from the control system. The transmitting device may be coupled to a plurality of medical devices and is configured to receive a plurality of status data from the plurality of medical devices.

In some embodiments, the control system is configured to output the signal to one or more of a speaker, a user interface, a storage device, an external device, a beacon, a writing device, a transmitting device, and an indicator.

In some embodiments, the medical device further includes a wake-up controller coupled to the control system, the wake-up controller configured to power on the medical device prior to control system transmitting a request for the status data.

In some embodiments, the control system is further configured to display on a user interface the status data via a display screen or a light indicator. The control system may be further configured to measure a current drawn from the electro-mechanical pneumatic system, and based on the current drawn from the electro-mechanical pneumatic system, determine a status of the electro-mechanical pneumatic system.

In some embodiments, the control system is further configured to measure a current

drawn from a speaker coupled to the control system, and based on the current drawn from the speaker, determine a status of the speaker.

In some embodiments, the control system is further configured to receive an input to power on the medical device, in response to the input, power on the medical device, and administer a status check to one or more of the electro-mechanical pneumatic system and the one or more components to obtain the status data of one or more of the electro-mechanical pneumatic system and the one or more components.

In some embodiments, the control system is configured to communicate with one or more other medical device units in a surrounding area to receive status data associated with the one or more other medical device units.

In some embodiments, the medical device further includes one or more accessories, wherein the control system is configured to receive accessory information associated with the one or more accessories.

In some embodiments, the control system includes a low power controller configured to transmit the request for the status data. The medical device may further include a beacon configured to provide an indication representative of the status data.

In some embodiments, the medical device further includes a power supply disposed within the housing and coupled to the control system, the power supply configured to receive a charge based on the status data.

In some embodiments, the electro-mechanical pneumatic system includes a blower having a fan, the fan being disposed within the blower.

In some embodiments, the one or more components include one or more of a power supply, a sensor, a valve, a wake-up controller, a speaker, and a user interface.

In some embodiments, the control system is further configured to power on the medical device prior to transmitting the request for status data, and power down the medical device upon outputting the signal based on the status data.

Another embodiment of the present disclosure may provide a medical device having a control system configured to without receiving an external request, automatically transmit to one or more components of the medical device a request for status data regarding a status of the one or more components, receive the status data from the one or more components, and output a signal based on the status data from the one or more components, wherein the signal is outputted to one or more of a speaker, a user interface, a storage device, an external device, a beacon, a writing device, a transmitting device, and an indicator.

In some embodiments, the control system is further configured to, based on the status data, provide a charge to a power supply disposed within the medical device.

Another embodiment of the present disclosure may provide a medical device including an electro-mechanical pneumatic system having a blower, the electro-mechanical pneumatic system disposed within the medical device and coupled to one or more components, and a controller coupled to the electro-mechanical pneumatic system and a control system, the controller configured to automatically power on the medical device, and transmit a signal to the control system to initiate a status check of one or more of the electro-mechanical pneumatic system and the one or more components of the medical device.

In some embodiments, the controller automatically powers on the medical device without receiving an external request.

### BRIEF DESCRIPTION OF THE DRAWINGS

The following detailed description of embodiments of the systems and methods of

administering a status check to a medical device will be better understood when read in conjunction with the appended drawings of an exemplary embodiment. It should be understood, however, that the invention is not limited to the precise arrangements and instrumentalities shown.

FIG. 1 is a top plan view of a ventilator system connected to a patient and having a medical device unit, a breathing circuit, and a patient interface in accordance with an exemplary embodiment of the present disclosure;

FIG. 2 is a schematic diagram of the ventilator system of FIG. 1;

FIG. 3 is a top perspective view of the medical device unit of FIG. 1;

FIG. 4 is a bottom view of the medical device unit of FIG. 1;

FIG. 5 is a front perspective view of the medical device unit of FIG. 1; and

FIG. 6 is a schematic diagram of the ventilator system of FIG. 1.

### DETAILED DESCRIPTION

Medical devices, such as ventilators, are commonly used for providing therapy and/or assistance to patients in respiratory distress. Most ventilators include many components working together to provide adequate breaths to a patient. During use or storage, one or more components of the ventilator can fail or malfunction resulting in a ventilator no longer operating correctly and becoming defective. A defective ventilator may not be identified prior to when it needs to be used, especially if the ventilator is stored with many other ventilators in a storage unit or a stockpile. Further, consistent testing and interaction with the ventilators to ensure that they are working correctly is time consuming.

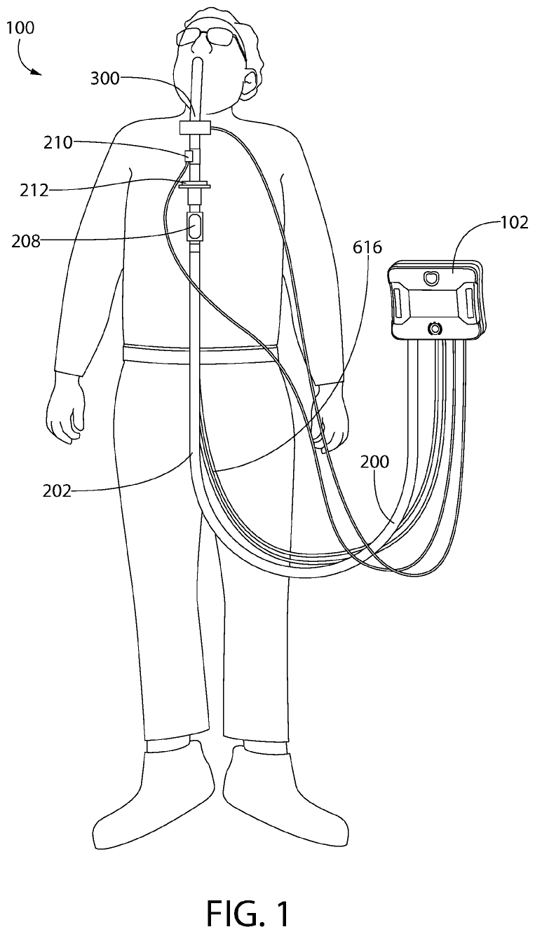

Exemplary embodiments of the present disclosure provide a system and method of administering a status check to a medical device. Embodiments of the present disclosure provide a system (e.g., ventilator system), generally designed 100, as shown in FIGS. 1-6. In use, system 100 may be used to provide assistance with breathing for the treatment of patients in a medical setting, such as the intensive care unit (ICU) of a hospital or a medical clinic. System 100 may also be used in other settings such as an ambulance, ambulatory center, in/out-patient centers, nursing homes/long term care facilities, and mobile clinics that can go to a patient directly. In some embodiments, system 100 is portable to allow for use in different environments. For example, system 100 may be easily transportable to be used in mobile settings (e.g., an ambulance). In some embodiments, system 100 includes a medical device unit being compact in size to allow for portability. For example, the medical device unit (e.g., ventilator) of system 100 may include a single circuit board containing all necessary and desired components to reduce the overall footprint of the medical device unit.

In some embodiments, system 100 allows for rapid initiation of emergency ventilation to a patient in respiratory distress. System 100 may be a rescue ready system configured to provide emergency treatment (e.g., ventilation) to a user or individual in respiratory distress. System 100 may be configured to provide rapid, emergency ventilation to a patient with minimal to no leakage of waste of air. System 100 may provide an efficient system for providing ventilation to a patient.

In use, system 100 may be used for the treatment of patients in a medical setting. For example, system 100 may be a ventilator to assist patients in respiratory distress or acute respiratory failure. System 100 may include medical device unit 102, breathing circuit 200, and patient interface 300. Medical device unit 102 may be configured to provide mechanical ventilation to a patient under respiratory failure through breathing circuit 200. Medical device unit 102 may provide the necessary gas flow or airflow, which may be directed through breathing circuit 200 to patient interface 300, which is coupled to the face of a patient. Medical device unit 102 may include blower 104, control system 106 and power supply 108. Breathing circuit 200 may include tube 202 which may be coupled to medical device unit 102 at first end 204 and coupled to patient interface 300 at second end 206.

In some embodiments, medical device unit 102 may be a ventilator used to provide assistance to a patient in respiratory distress. Medical device unit 102 may be configured to provide different modes of ventilation to a patient. For example, medical device unit 102 may be configured to provide assist-controlled ventilation, volume-controlled ventilation, pressure support, pressure-controlled ventilation, pressure regulated volume control, positive end expiatory pressure, synchronized intermittent-mandatory ventilation, and/or manual ventilation. Medical device unit 102 may be used instead of a bag valve device, an emergency transport ventilator, or any other modes or devices for providing ventilation to a patient.

Medical device unit 102 may be configured to perform a self-test (e.g., status check) when stored or in storage, during start-up, or during use. A self-test may include a control system transmitting one or more signals to various components of medical device unit 102 to determine whether they are operating correctly. For example, medical device unit 102 may perform a self-test, which causes one or more components of medical device unit 102 to go through calibrations and checks to ensure that they are performing optimally or within specific parameters based on the desired use of medical device unit 102. The one or more components may include sensors, valves, resistors, capacitors, transistors, power supplies (e.g., power supply 108), controllers, logic boards, ports, user interfaces, and/or electro-mechanical pneumatic systems (e.g., blower 104).

In some embodiments, medical device unit 102 performs a self-test upon powering on. Medical device unit 102 may perform a self-test or status check automatically without being interrogated or requested to do perform a self-test or status check (e.g., by a user or external device). However, medical device unit 102 may be configured to perform a self-test upon a request received from a remote device or server or via polling from an external device. In some embodiments, medical device unit 102 is configured to perform a self-test upon passive interrogation. For example, medical device unit 102 may perform a self-test based on receiving a signal without sending a request for the signal.

In some embodiments, medical device unit 102 is configured to perform a self-test of one or more components of medical device unit 102 upon initial start-up (e.g., boot-up) and/or upon restarting of medical device unit 102. The same components of medical device unit 102 may be tested during start-up and restart. However, medical device unit 102 may perform self-tests on different components based on whether medical device unit 102 is starting up or restarting. For example, upon start-up, medical device unit 102 may perform a self-test on a first set of components. Upon restart, medical device unit 102 may perform a self-test on a second set of components. The first set of components and the second set of components may be different, the same, or including overlapping components. In some embodiments, upon start-up, medical device unit 102 performs a self-test on a greater number of components than when a self-test is performed during a restart. For example, during start-up, medical device unit 102 may perform a self-test on all or substantially all components of medical device unit 102 that are configured to be tested compared to a restart, where less than all or only a few components of medical device unit 102 are tested. In some embodiments, on start-up, medical device unit 102 is in a new state with default settings (e.g., new patient settings). During restart, medical device unit 102 may load with settings that it previously had prior to restarting (e.g., not the default settings).

In some embodiments, medical device unit 102 includes a button that when actuated causes medical device unit 102 to perform a self-test. In some embodiments, medical device unit 102 is configured to perform a self-test without coupling to any other device. For example, in use medical device unit 102 may couple to patient interface 300 via breathing circuit 200. Medical device unit 102 may be configured to perform a self-test or status check prior to coupling to breathing circuit 200. In some embodiments, medical device unit 102 is configured to perform a self-test without having to couple to breathing circuit 200 and/or patient interface 300.

Referring to FIGS. 1-5, medical device unit 102 may include housing 132, blower 104,

control system 106, and power supply 108. Housing 132 of medical device unit 102 may house and protect the components (e.g., sensors, valves, resistors, capacitors, transistors, power supplies (e.g., power supply 108), controllers, logic boards, ports, and/or electro-mechanical pneumatic systems (e.g., blower 104)) disposed within medical device unit 102. Housing 132 may be lightweight to allow for easy portability of medical device unit 102. For example, housing 132 of medical device unit 102 may be made of a lightweight polymer to allow for easy transportation. In some embodiments, housing 132 is comprised of one or more of acrylonitrile butadiene styrene acrylonitrile butadiene styrene (ABS), polyoxymethylene (POM), aliphatic polyamides (PPA), polycarbonate (PC), polyphenylsulfone (PPSU), polyetherimide (PEI), and polypropylene (PP). Housing 132 may be comprised of a lightweight, but durable material to allow for repeated use in harsh environments, while still providing portability. For example, housing 132 may be comprised of ABS to provide portability and to ensure that the components disposed within housing 132 are secured, protected, and remain undamaged. In some embodiments, housing 132 of medical device unit 102 is substantially rectangular shaped to allow for easy storage. However, housing 132 may be square, circular, triangle, octagonal, or any other shape desired. In some embodiments, housing 132 includes sidewalls 130. In a preferred embodiment, housing 132 includes four sidewalls 130 to define a substantially rectangular shape of medical device unit 102. In some embodiments, housing 132 has rounded corners and beveled edges to allow for a more ergonomic shape.

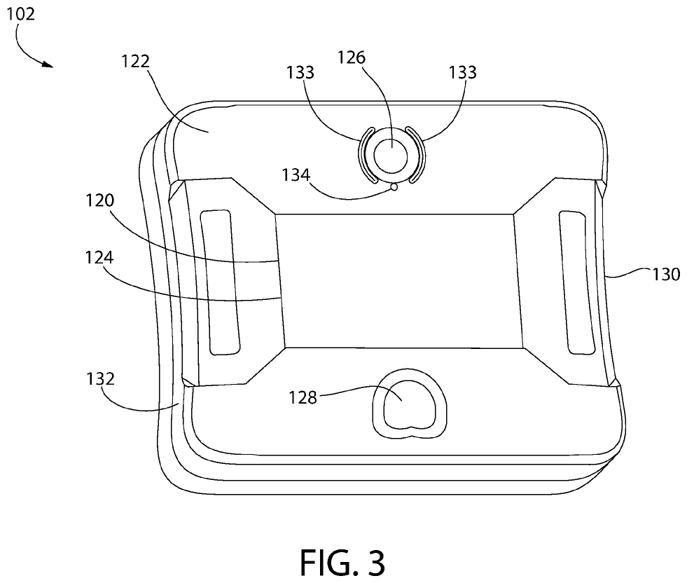

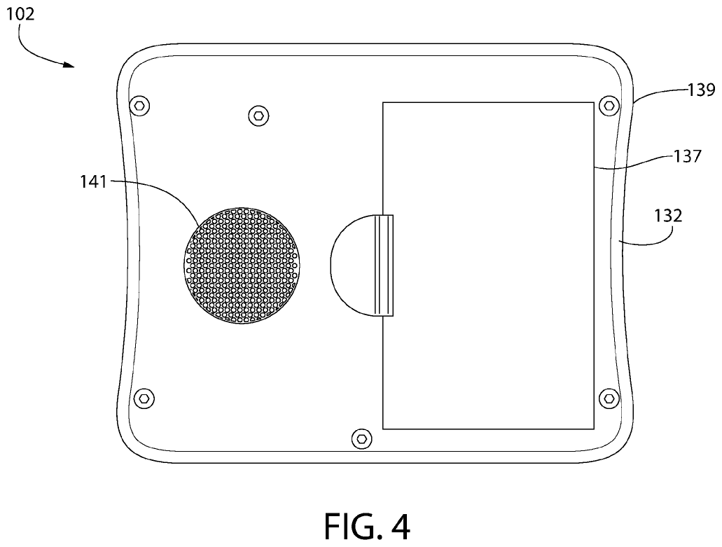

Referring to FIGS. 3-4, housing 132 may include top surface 122 and bottom surface 139. In some embodiments, top surface 122 is parallel to bottom surface 139. Top surface 122 may be coupled to bottom surface 139 via sidewalls 130. Housing 132 may include cutout 120 disposed on top surface 122 of housing 132. Cutout 120 may be sized and shaped to receive user interface 124. User interface 124 may be a display device, which may be disposed within cutout 120, and may be configured to receive input from a user. In some embodiments, user interface 124 is a graphical user interface. For example, user interface 124 may be a touch screen configured to receive inputs from a user and transmits the inputs to control system 106. In some embodiments, a user interacts with medical device unit 102 (e.g., via user interface 124) to power on or power off medical device unit 102. For example, control system 106 may receive an input from a user to power on medical device unit 102 and in response to that input, control system 106 may power on medical device unit 102 and administer a status check or self-test. Further, user interface 124 may be used to display information about a patient using medical device unit 102. For example, user interface 124 may display an indication of the respiratory status of a patient coupled to patient interface 300.

In some embodiments, user interface 124 may various settings, parameters, and/or

functionalities of the components disposed within medical device unit 102. For example, user interface 124 may display the peak inspiratory pressure (PIP), tidal volume (TV), respiratory rate (RR), positive end expiratory pressure (PEEP), inspiratory to expiatory ratio (I:E ratio), ventilation mode, peak flow, and sensitivity. User interface 124 may be coupled to control system 106 and may be configured to control various components of system 100. For example, a user may interact with user interface 124 to change parameters of blower 104.

In some embodiments, medical device unit 102 includes speaker 141. Speaker 141 and/or user interface 124 may be configured to provide instructions and/or alerts to the user. For example, user interface 124 may provide visual instructions to a user for correcting an error to medical device unit 102 (e.g., replacing or fixing one or more components, removing occlusions, changing settings) and speaker 141 may provide audio instructions to a user for correcting the error to medical device unit 102. In some embodiments, user interface 124 is configured to display a video or graphics to a user to instruct them on how to fix or address an error to medical device unit 102. In some embodiments, speaker 141 is configured to provide an audio alert or alarm to a user based on an error detected by medical device unit 102. User interface 124 may be configured to provide a visual alert or alarm to a user based on an error detected by medical device unit 102. In some embodiments, medical device unit 102 includes a vibrator such that when an error occurs, medical device unit 102 vibrates or provides other haptic feedbacks. Medical device unit 102 may transmit an outgoing signal when an alert occurs to alert a remote user.

In some embodiments, a user interacts with user interface 124 to change various operating modes and/or parameters of medical device unit 102. For example, user interface 124 may provide an option for adjusting the PEEP, the PIP, the tidal volume, the I:E ratio, or other parameters.

In some embodiments, medical device unit 102 includes beacon or indicator 134 to provide a status of system 100. Indicator 134 may provide the status of system 100 and/or medical device unit 102. For example, indicator 134 may indicate whether medical device unit 102 is damaged, inoperable, and/or functionally properly. Indicator 134 may be an LED and control system 106 may transmit a status to indicator 134 causing indicator 134 to illuminate a specific color and flash at specific frequency. For example, indicator 134 may be a visual indicator, such as an LED, indicating that status of medical device unit 102. In some embodiments, indicator 134 continuously provides a visual indication that medical device unit 102 is functioning properly and an interruption in the visual indication indicates that an error has occurred with medical device unit 102. In some embodiments, medical device unit 102 includes a beacon in addition to or alternative to indicator 134. The beacon may be configured to transmit a signal (e.g., wireless signal) regarding the status of medical device unit 102.

Indicator 134 may be a transmitter configured to transmit an outgoing signal. In some embodiments, indicator 134 is configured to continuously transmit an outgoing signal regarding the status of medical device unit 102. For example, indicator 134 may be configured to continuously transmit a signal without be requested to transmit a signal. Indicator 134 may transmit a signal indicating all components of medical device unit 102 are functioning correctly. Indicator 134 may be configured to output a signal, such as a radio frequency (RF) signal indicating the status of medical device unit 102.

In some embodiments, indicator 134 is configured to transmit a continuous signal (e.g., continuous RF signal) indicating that medical device unit 102 is operating correctly. An interruption in the continuous signal may indicate an error with medical device unit 102. In some embodiments, indicator 134 continuously transmits a signal until an error occurs, which interrupts the signal transmission resulting in indicator 134 no longer transmitting a signal. A user may check a receiver to determine whether indicator 134 is transmitting a signal and whether an error has occurred based on the transmission ceasing. In other embodiments, indicator 134 is configured to transmit a first signal when medical device unit 102 is functioning correctly without significant errors (e.g., blower 104 failing or occluding, loss of power to one or more components, excess build-up of oxygen) and is configured to transmit a second signal when an error occurs. The first signal may be different than the second signal. Indicator 134 may transmit a signal wirelessly via radio frequency, WiFi, cellular signal, Bluetooth, near field communication (NFC), or any other type of wireless modality.

In some embodiments, indicator 134 provides a status of medical device unit 102 without requiring a user to interact with or power on medical device unit. For example, indicator 134 may be coupled to a power supply separate from power supply 108 and may be configured to illuminate to provide an indication of a status to a user without the user interacting with medical device unit 102. Indicator 134 may transmit a signal to an external receiving device. In some embodiments, indicator 134 transmits a signal regardless of whether an external receiving device is proximate to medical device unit 102 or whether an external receiving device is requesting data from indicator 134. For example, indicator 134 may be configured to transmit a signal regardless of whether a device is listening or whether a device is requesting a signal from indicator 134. In some embodiments, indicator 134 is configured to always be transmitting a signal when medical device unit 102 is functioning correctly or operating normally.

In some embodiments, indicator 134 is configured to flash different colors of light. For example, indicator 134 may flash the color green when medical device unit 102 is operating normally, flash the color red when medical device unit 102 is malfunctioning, or flash the color yellow when medical device unit 102 has an error, but can still function. However, indicator 134 may flash or have a constant illumination. Indicator 134 may be any color desired and may alternate between different colors depending on the status of medical device unit 102. In some embodiments, indicator 134 is coupled to a power supply to ensure that indicator 134 is able to continuously provide an indication for the status of medical device unit 102.

In practice, control system 106 may perform a self-test or status check without user intervention and may cause indicator 134 to illuminate based on the results of the self-test or status check (e.g., status data). A user may view medical device unit 102 after the self-test or status check has been performed and may view indicator 134. Upon viewing indicator 134, a user may be able to determine the status of medical device unit 102 and if there are any errors associated with medical device unit 102 without having to interact with medical device unit 102. Interacting with medical device unit 102 may including actuating one or more buttons on medical device unit 102, powering on medical device unit 102, or engaging with user interface 124. In practice, a user may view indicator 134 immediately after the self-test or status check has been performed or may view indicator 134 after a duration of time as elapsed since the self-test or status check has been performed. In some embodiments, control system 106 is configured to transmit a signal to indicator 134 regardless of the power status of medical device unit 102. In other words, indicator 134 may be configured to always receive a signal from control system 106 regardless of the power status of medical device unit 102. This may be due to control system 106 and indicator 134 each having their own power supply separate from power supply 108 or control system 106 and indicator 134 sharing a power supply separate from power supply 108. In some embodiments, indicator 134 has a low power sensor configured to receive a signal from control system 106 to illuminate based on the status of a performed self-test or status check.

In some embodiments, housing 132 also includes indicator 133. Indicator 133 may be similar to indicator 134. Indicator 133 may also indicate the status of medical device unit 102 and may be used to provide alerts to the user regarding an alarm condition. For example, indicator 133 being green may indicate normal operation of medical device unit 102. However, indicator 133 flashing amber, red, yellow, or orange may indicate a malfunction or error with medical device unit 102. In some embodiments, the degree of flashing of indicator 133 indicates the severity of the error. Indicator 134 may also indicate the battery status associated with power supply 108. For example, indicator 134 being green may indicate that the battery of medical device unit 102 is fully charged. Indicator 134 being other colors, such as red, orange, yellow, amber, and/or flashing may indicate a malfunction or power level of the battery.

Medical device unit 102 may include one or more buttons that control system 100. For example, medical device unit 102 may include buttons 126 and 128, which control the power status and functions of medical device unit 102. In some embodiments, button 126 is a power ON/OFF button to control the power status of medical device unit 102. For example, a user may press button 126 to power on medical device unit 102. Button 128 may be a manual breath button, which delivers a single breath at a predetermined tidal volume to a patient. In some embodiments, button 128 may need to be pressed for a predetermined amount of time before medical device unit 102 delivers a single breath to the patient.

Referring to FIGS. 1-5, medical device unit 102 may include pneumatic system or blower 104, which may include motor 110 and fan 112. In some embodiments, pneumatic system 104 is an electro-mechanical pneumatic system. Pneumatic system or blower 104 may include any system configured to generate air flow and/or pressure such as a compressor, high pressure gas source, piston, or fan.

Motor 110 may be coupled to fan 112 and motor 110 may be configured to rotate fan 112 to generate air flow. In some embodiments, motor 110 is configured to rotate fan 112 at maximum of 37,500 revolutions per minute (RPM). Fan 112 may rotate to generate airflow that exits blower 104. Motor 110 may be coupled to control system 106, which may control motor 110. In some embodiments, fan 112 is configured to provide a maximum of 1,000 liters per minute (LPM). In some embodiments, fan 112 is configured to rotate at greater than 37,500 RPMs and greater than 1,000 LPMs.

In some embodiments, blower 104 may be disposed within enclosure 114. Enclosure 114 may be sized and shaped to receive blower 104 and may be a unitary piece. For example, enclosure 114 may be comprised of two halves and may be configured to receive blower 104 such that blower 104 is disposed within enclosure 114. Enclosure 114 being made comprised of two halves which surround blower 104 allows for the reduction of components and material needed to manufacture system 100. Blower 104 may include an inflow, which may be disposed within enclosure 114. In some embodiments, the inflow of blower 104 may be the only portion of blower 104 disposed within enclosure 114.

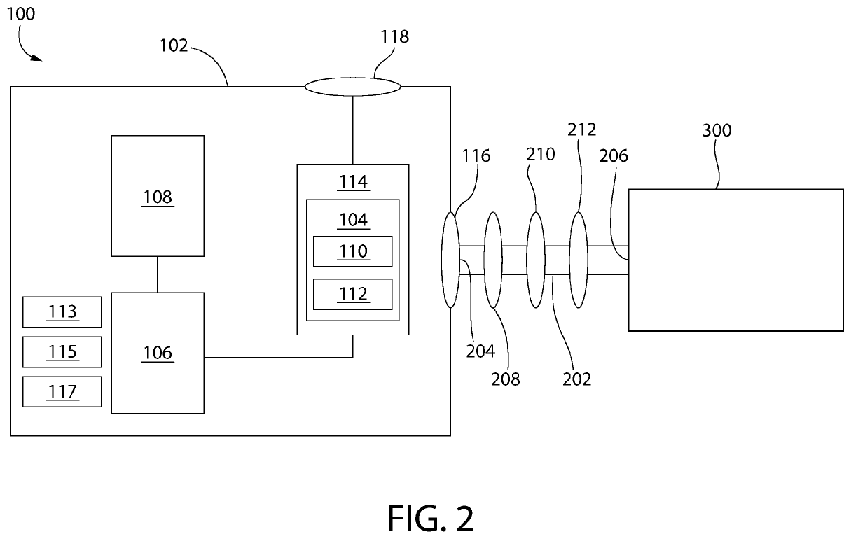

Referring to FIGS. 1-2 medical device unit 102 may include control system 106. Control system 106 may be a microcontroller, a peripheral interface controller (PIC), a system on a chip (SoC), or a processor. In some embodiments, control system 106 is a low power controller. For example, control system 106 may be a low power controller coupled to a power supply such that control system 106 is configured to run for extended period of time (e.g., several years). Control system 106 may be coupled to one or more components of system 100 (e.g., sensors, valves, resistors, capacitors, transistors, power supplies (e.g., power supply 108), controllers, logic boards, ports, user interfaces, and/or electro-mechanical pneumatic systems (e.g., blower 104)). In some embodiments, control system 106 is coupled to blower 104 to control motor 110, which controls fan 112. In some embodiments, control system 106 controls the volume of gas delivered to a patient by attenuating the speed of fan 112. For example, controls system 106 may attenuate the power delivered to motor 110, thereby decreasing the speed of fan 112 to reach a target amount of gas delivered to a patient through breathing circuit 200. Control system 106 may include writing device 113, which may be configured to write information to transmitting devices 117, such as radio-frequency identification (RFID) chips/tags. In some embodiments, control system 106 is coupled to power supply 108. However, control system 106 may be coupled to its own power supply. Control system 106 may include software configured to control control system 106 and one or more components coupled to control system 106. The software may be updated from a remote server or external device.

In some embodiments, writing device 113 is disposed within medical device unit 102. However, writing device 113 may be disposed outside of medical device unit 102 and may be an external device. Writing device 113 may be disposed within, on, or outside of medical device unit 102 and may wirelessly communicate with transmitting device 117. In some embodiments, writing device 113 is configured to wirelessly write information to transmitting devices 117. Writing device 113 may be coupled to control system 106 and may be stored anywhere within medical device unit 102. Writing device 113 may further be coupled to memory 115, which may be coupled to control system 106.

In some embodiments, transmitting device 117 is stored within medical device unit 102 and is communicatively coupled to control system 106. However, transmitting device 117 may be disposed on or near housing 132 of medical device unit 102 and may be configured to wirelessly communicate with control system 106. For example, transmitting device 117 may be coupled to the exterior surface of housing 132 and may wirelessly receive information from control system 106. Transmitting device 117 may be a storage device configured to wirelessly transmit information, such as a wireless transmitting device. For example, transmitting device 117 may include one or more of an RFID chip/tag, a near-field communication chip, a Bluetooth transmitter, a digital barcode, QR code, or a WiFi module. In some embodiments, user interface 124 is configured to display the digital barcode or QR code such that a user can scan the digital barcode or QR code to retrieve status data of one or more medical device unit 102. In some embodiments, transmitting device 117 transmits information upon request. However, transmitting device 117 may be configured to transmit information associated with a self-test automatically and/or autonomously without intervention by a user or external device, or without receiving a request to transmit information. Transmitting device 117 may be configured for low-power consumption. In some embodiments, transmitting device 117 is configured to receive power only from an external source. However, transmitting device 117 may be powered by power supply 108 or its own power supply.

Control system 106 may receive information associated with, for example, the status of system 100 and store the information in memory 115 or directly to transmitting device 117. Writing device 113 may access memory 115 and may write the information stored within memory 115 to transmitting device 117. In some embodiments, memory 115 includes transmitting device 117. Memory 115 may include, for example, random access memory (RAM), a hard disk drive and/or a removable storage drive, such as a floppy disk drive, a magnetic tape drive, an optical disk drive, or a wireless device, such as an RFID tag. Memory 115 may include other similar means for allowing computer programs or other instructions to be loaded into system 100. For example, memory 115 may include a removable memory chip (such as an EPROM, or PROM, or flash memory) and associated socket, and other removable storage units and interfaces which allow software and data to be transferred from a removable storage unit to system 100. In some embodiments, memory 115 is a non-volatile memory. In some embodiments, memory 115 is configured for low-power consumption or configured to receive power only from an external source.

In some embodiments, control system 106 is coupled to power supply 108, which may be configured to provide power to the various components of system 100. For example, control system 106 may be configured to route power from power supply 108 to motor 110 of blower 104. Power supply 108 may be disposed within medical device unit 102. Power supply 108 may include one or more of an internal rechargeable battery, a removable rechargeable battery, and a removable non-rechargeable battery. In some embodiments, control system 106 is coupled to power supply separate from power supply 108.

In some embodiments, power supply 108 is configured to be recharged using an external device. For example, power supply 108 may be recharged by plugging medica device unit 102 into an electrical outlet. Power supply 108 may be recharged wirelessly. In some embodiments, each power supply 108 of a plurality of medical device units 102 are configured to be recharged via a wired connection or wirelessly. Multiple power supplies 108 each belonging to medical device unit 102 may be charged simultaneously. For example, multiple power supplies 108 of a plurality of medical device 102 may be charged wirelessly by a wireless charging device disposed proximate to the plurality of medical devices 102. In some embodiments, one medical device 102 is configured to charged or re-charged an adjacent medical device 102.

In some embodiments, medical device unit 102 is configured to wake up and administer a status check. The status check may determine that power supply 108 of medical device 102 has a low charge (e.g., a charge less than a predetermined minimum charge threshold). In response to determining that power supply 108 has a low charge, power supply 108 may begin charging until power supply 108 is at a full charge or reached a predetermined maximum charge threshold. In some embodiments, medical device unit 102 is coupled to an electrical outlet or disposed proximate a wireless charging device such that power supply 108 can be re-charged when power supply 108 has a low charge.

In practice, medical device unit 102 is coupled to an electrical outlet or disposed proximate a wireless charging device and control system 106 controls when power supply 108 receives a charge. Control system 106 may initiate charging of power supply 108 (e.g., allow current to flow to power supply 108) when power supply 108 has a low charge (e.g., a charge less than a predetermined minimum charge threshold) and may cease charging of power supply 108 (e.g., prevent current to flow to power supply 108) when power supply 108 is at a full charge or reached a predetermined maximum charge threshold. Medical device unit 102 may be configured to wake up, administer a status check, and automatically (e.g., without human intervention) charge/re-charge power supply 108 based on the status of power supply 108. Power supply 108 may be charged/re-charged due to medical device unit 102 being coupled to an electrical outlet or being disposed proximate a wireless charging device. This allows medical device unit 102 to be stored or stockpiled for a long duration without power supply 108 being depleted or having a low charge upon use.

In some embodiments, the predetermined minimum charge threshold is 15%. However, the predetermined minimum charge threshold may be 0% to 50%, 3% to 40%, 5% to 30%, or 10% to 25%. The predetermined minimum charge threshold may be changed by a user. For example, a user may interact with user interface 124 to adjust or change the predetermined minimum charge threshold. In some embodiments, the predetermined minimum charge threshold is changed remotely (e.g., via an external device or a software update).

The predetermined minimum charge threshold may be changed based on the health of power supply 108. For example, due to several charges/re-charges, the health of power supply 108 may deteriorate resulting in a deteriorated power supply 108 lasting a shorter duration compared to a fully health power supply 108. The predetermined minimum charge threshold may vary based on the health of power supply 108 such that power supply 108 is able to provide power to medical device unit 102 for a sufficient amount of time. For example, a deteriorated power supply 108 that has a charge of 50% may last the same amount of time as a fully healthy power supply 108 that has a charge of 5%. The status check performed on medical device unit 102 may provide information about the health of power supply 108 to allow control system 106 to determine the predetermined minimum charge threshold and provide charge to power supply 108 accordingly.

In some embodiments, the predetermined maximum charge threshold is 100%. However, the predetermined maximum charge threshold may be 50% to 100%, 55% to 95%, 60% to 90%, or 75% to 80%. The predetermined maximum charge threshold may be changed by a user. For example, a user may interact with user interface 124 to adjust or change the predetermined maximum charge threshold. In some embodiments, the predetermined maximum charge threshold is changed remotely (e.g., via an external device or a software update). The predetermined maximum charge threshold may be changed to conserve the health and longevity of power supply 108. For example, consistent charging/re-charging of power supply 108 to 100% may reduce the overall health of power supply 108 resulting in deterioration of the health of power supply 108. Charging power supply 108 to less than 100% (e.g., 80%) may lengthen the lifespan of power supply 108.

As shown in FIG. 4, medical device unit 102 may be configured to receive a battery pack via battery storage 137. In some embodiments, a user may place a removable rechargeable battery and/or a removable non-rechargeable battery within battery storage 137. In some embodiments, power supply 108 may be coupled to a power source (not shown) via a power adapter. Power supply 108 may control the voltage and current from a power source to control system 106.

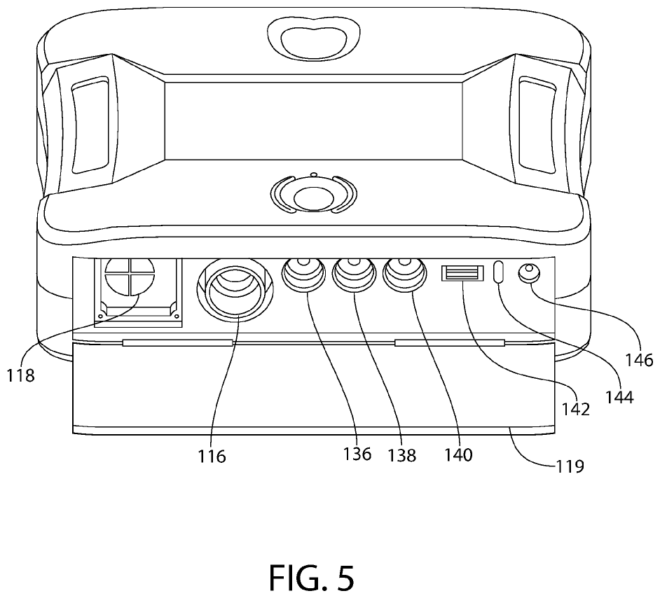

Referring to FIG. 5, in some embodiments, medical device unit 102 may include inlet 118 and outlet 116. Inlet 118 may be disposed on one of sidewalls 130 of housing 132 and allow for air to flow from the external environment (ambient air) or an air source, such as a reservoir of gas (O₂), to blower 104. For example, blower 104 may be configured to pull in air from inlet 118 and push the air out through outlet 116. In some embodiments, medical device unit 102 relies on blower 104 to provide air and does not require compressed air to operate. In some embodiments, blower 104 is coupled to outlet 116, which is disposed on an outer periphery of housing 132. For example, outlet 116 may be disposed on sidewall 130 of housing 132. Outlet 116 may be cylindrical in shape and hollow. In some embodiments, outlet 116 couples blower 104 to breathing circuit 200 to patient interface 300. For example, outlet 116 may be configured to allow air to flow from blower 104 of medical device unit 102 through breathing circuit 200 to patient interface 300. In some embodiments, outlet 116 is a valve that may open or close to control the airflow from blower 104 to breathing circuit 200. Outlet 116 may be controlled by air pressure or by control system 106.

Referring to FIGS. 1-2, system 100 may include breathing circuit 200. Breathing circuit 200 may be coupled to medical device unit 102. For example, breathing circuit 200 may be coupled to outlet 116. In some embodiments, breathing circuit 200 may be disposed between medical device unit 102 and patient interface 300. Breathing circuit 200 may be configured to receive air from medical device unit 102. Breathing circuit 200 may include tube 202, exhale valve 208, flow sensor 210, and patient filter 212. Tube 202 may include first end 204 and second end 206. First end 204 may be coupled to medical device unit 102 and second end 206 may be coupled to patient interface 300. In some embodiments, tube 202 is a cylindrical lumen configured to allow airflow from medical device unit 102 to patient interface 300. Tube 202 may be configured to include exhale valve 208, flow sensor 210, and patient filter 212. Exhale valve 208 disposed on or within tube 202 and may be configured to open on exhalation of the patient using system 100 to allow air to flow out of the patient. Exhale valve 208 may be closed during inhalation such that air does not exist system 100, thereby increasing efficiency. For example, exhale valve 208 may be closed during inhalation to ensure that the proper amount and flow of air reaches patient interface 300.

In some embodiments, exhale valve 208 is controlled by control system 106 to control the exhalation of the patient. In another embodiment, exhale valve 208 is controlled based on the exhalation of the patient. In yet another embodiment, exhale valve is controlled by both control system 106 and the exhalation of the patient. Exhale valve 208 may be configured to allow for a specific respiration rate but may be opened by the exhalation of the patient as well. For example, for a respiration rate of 12 (one breathe ever five seconds), exhale valve 208 may open ever five seconds and may also open more than every five seconds if the patient is breathing at different rate.

In some embodiments, breathing circuit 200 includes flow sensor 210, which may be disposed on or within tube 202. Flow sensor 210 may be configured to sense the flow of air within breathing circuit 200. For example, flow sensor 210 may detect the rate and amount of air flowing through tube 202. In some embodiments, flow sensor 210 is coupled to control system 106 to provide feedback to system 100. For example, flow sensor 210 may provide information to control system 106, which may change the parameters of blower 104 based on the information.

Breathing circuit 200 may further include patient filter 212, which may be disposed proximate second end 206 of tube 202. For example, patient filter 212 may be disposed on or within tube 202 proximate second end 206 and adjacent to patient interface 300. Patient filter 212 may be configured to filter out particles within air. For example, patient filter 212 may filter out particles and airborne viruses to protect the patient using system 100.

Referring to FIGS. 1-2, system 100 may include patient interface 300. Patient interface 300 may be a device that is secured to the face of a patient. For example, patient interface 300 may be a bag valve mask, respirator, or an endotracheal (ET) tube used for intubation.

Referring to FIG. 5, medical device unit 102 may further included various inputs for coupling medical device unit 102 to other components of system 100. In addition to inlet 118 and outlet 116, medical device unit 102 may include control line port 136, pressure line port 138, differential pressure tube port 140, flow sensor port 142, data communication port 144, and power port 146. Control line port 136 may be used to couple exhale valve 208 and medical device unit 102. For example, exhale valve 208 may be coupled to medical device unit 102 at control line port 136 such that medical device unit 102 can control the opening and closing of exhale valve 208. Pressure line port 138 and differential pressure tube port 140 may be used to couple one or more pressure sensors to medical device unit 102. Flow sensor port 142 may be used to couple flow sensor 210 to medical device unit 102. For example, flow sensor 210 may be coupled to medical device unit 102 at flow sensor port 142 such that medical device unit 102 can receive information from flow sensor 210. Data communication port 144 may be used to couple medical device unit 102 to an electronic device such as a computer system, a mobile device, a server, etc. Power port 146 may be used to couple medical device unit 102 to a power source. For example, power port 146 may be configured to couple power supply 108 to a power source to provide power to medical device unit 102 through power supply 108.

Medical device unit 102 may include port plate 119. Port plate 119 may be a portion of

housing 132 that protects one or more of inlet 118, outlet 116, control line port 136, pressure line port 138, differential pressure tube port 140, flow sensor port 142, data communication port 144, and power port 146. Port plate 119 may be configured to prevent debris from entering the ports of medical device unit 102. In some embodiments, port plate 119 includes one or more filters to filter air/gas entering through various inlets of medical device unit 102. Port plate 119 may be hingedly coupled to housing 132. In some embodiments, port plate 119 is a separate component from housing 132 and may be slidably received by housing 132 adjacent to the ports of medical device unit 102. For example, port plate 119 may be molded to housing 132 and may be manufactured via injection molding.

Inlet 118 may include cover or door 121 disposed over inlet 118. Cover 121 may be configured to allow inlet 118 to be connected to air/gas source, such as an oxygen source. Inlet 118 may also include cover 121 to prevent connection of the wrong connector to inlet 118. For example, inlet 118 may include a specialized cover configured to allow only for reservoirs of only certain gases or fluids to flow into inlet 118. In some embodiments, cover 121 may prevent inadvertent connection of breathing circuit 200 to the wrong connection. In some embodiments, a user would have to actively remove cover 121 from inlet 118 to allow connection of an air/gas source to inlet 118. In some embodiments, cover 121 may be coupled to port plate 119. For example, cover 121 may be hingedly coupled to port plate 119 to allow for covering of inlet 118. In some embodiments, cover 121 may allow ambient air to flow into inlet 118 without removing cover 121 from inlet 118. In some embodiments, cover 121 may have special markings to indicate which sources of air/gas can be coupled to inlet 118. In some embodiments, a special tool is required to remove cover 121 from inlet 118 to prevent inadvertent connection to inlet 118. In some embodiments, cover 121 includes a sensor to only allow removal from inlet 118 when certain gases are detected. Cover 121 may also be configured to prevent debris from entering inlet 118.

Medical device unit 102 may include a testing connection configured to allow for the testing of airflow and blower 104 of medical device unit 102. In some embodiments, the testing connection is disposed on or within port plate 119. However, the testing connection may be disposed on housing 132. In some embodiments, the testing connection is disposed on port plate 119 and allows air coming from outlet 116 of fan 112 to flow through the testing connection into a pressure sensor disposed on port plate 119 or the testing connection. For example, the testing connection may include a recess that allows air to flow from outlet 116 to the pressure sensor to determine the pressure of air provided by blower 104. The recess of the testing connection may allow for air to be channeled from outlet 116 to the pressure sensor, which may be disposed on port plate 119 or housing 132. In some embodiments, the testing connection connects outlet 116 to a pressure sensor or pressure line to test the pressure of the air from outlet 116. Testing connection may provide additional protection to port plate 119 and outlet 116. The testing connection may be a tube coupling outlet 116 to the pressure sensor and/or pressure line. In some embodiments, the tube is disposed on housing 132. The testing connection may allow for testing of blower 104 when medical device unit 102 is in storage. The testing connection may be configured to ensure the integrity of pressure sensors of medical device unit 102. In some embodiments, the recess of testing connection may allow air to flow from outlet 116 to other sensors disposed on port plate 119 and/or within the testing connection. The testing connection may be hingedly coupled to port plate 119 or housing 132 and may be configured to be completely removable from medical device unit 102.

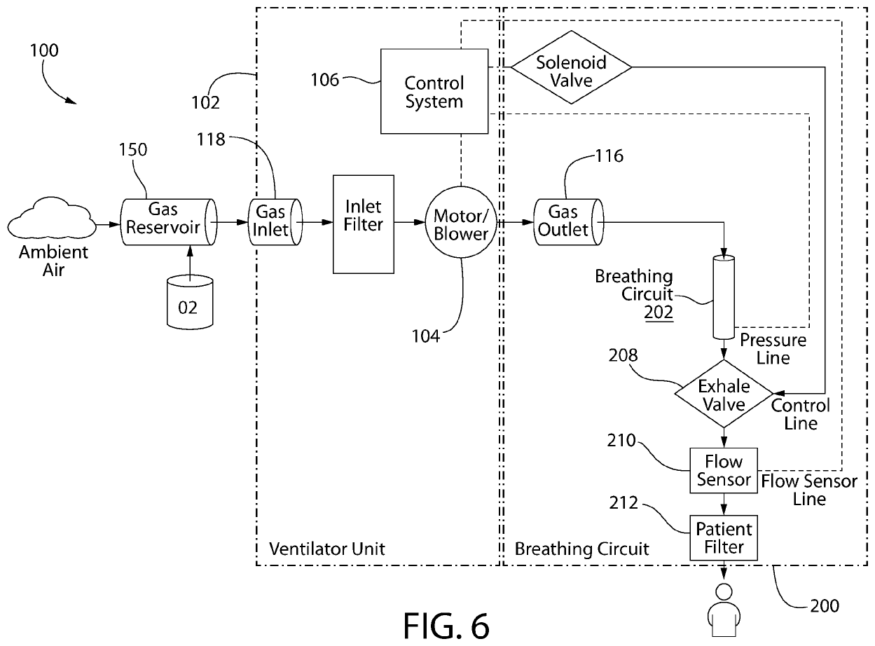

Referring to FIG. 6, ambient air and oxygen may both enter gas reservoir 150 and mix together. Gas from gas reservoir 150 may enter medical device unit 102 through inlet 118 to prevent external debris from entering medical device unit 102. The gas is then channeled through an air pathway housed in medical device unit 102, and into breathing circuit 200 through outlet 116.

In some embodiments, system 100 may be configured to administer a status check or a

self-test to ensure that some or all of the components are working properly and that there are not any malfunctions. In some embodiments, control system 106 is configured to test the various components of system 100 to determine the functional status of, for example, blower 104, power supply 108, writing device 113, memory 115, transmitting device 117, and control system 106, in addition to reporting the operational status of system 100. For example, control system 106 may be configured to receive information from memory 115 regarding any corrupted cores, from blower 104 regarding an occlusion of fan 112, from outlet 116 or inlet 118 regarding occlusions, from power supply 108 regarding improper voltages, or any other information necessary to ensure that medical device unit 102 is functioning properly. Control system 106 may be configured to perform one or more self-tests on one or more components including a system start-up test, a motor test, a user interface test, a button test, a temperature sensor test, a motor voltage test, a motor current test, a motor initiation test, a patient pressure test, a blower pressure test, an oxygen sensor test, an ambient sensor test, a barometer test, a speaker test, a system fatal error test, and/or a software test.

In some embodiments, control system 106 automatically receives information from various components of system 100 on a periodic or aperiodic basis. For example, control system 106 may receive information for some or all of the components of medical device unit 102 without receiving a request from a user or other device. However, control system 106 may receive a request from a user to perform a self-test on one or more components of medical device unit 102.

In some embodiments, medical device unit 102 includes a wake-up controller. The wake-up controller may be communicatively coupled to control system 106. The wake-up controller may be configured to wake-up (e.g., power on) medical device unit 102 to allow control system 106 to perform a self-test. In some embodiments, the wake-up controller is a microcontroller coupled to a timer. The timer may be configured to power on medical device unit 102 via the wake-up controller. The timer may transmit a signal to wake-up controller on a periodic basis, an aperiodic basis, a random basis, or a pre-programmed basis to power on medical device unit 102. The wake-up controller may include a clock or timer. In some embodiments, the wake-up controller is configured to receive a request to perform a self-test. Upon receiving the signal, wake-up controller may power on medical device unit 102 and instruct control system 106 to perform a self-test on medical device unit 102. In some embodiments, wake-up controller and control system 106 are coupled to different power supplies. For example, wake-up controller may be coupled to power supply 108 (e.g., a battery) and control system 106 may be coupled to a different power supply and configured to run on a small amount of power. In some embodiments, control system 106 is coupled to a small battery configured to output little power and have longevity. Control system 106 may be coupled to a small battery due to performing a self-test requiring low power consumption. In some embodiments, control system 106 and the wake-up controller are coupled to the same power supply. In some embodiments, wake-up controller is a low power controller. For example, wake-up controller may be a low power controller coupled to a power supply such that wake-up controller is configured to run for extended periods of time (e.g., several years).

In some embodiments, control system 106 is configured to perform a self-test on a daily basis, weekly basis, monthly basis, bi-monthly basis, or any other amount of repeating time. Control system 106 may be configured to test certain components more frequently than other components. For example, control system 106 may be configured to test blower 104 on a monthly basis and speaker 141 on a daily basis. By way of another example, control system 106 may be configured to test all components sequentially or simultaneously on a monthly basis and test individual components (e.g., blower 104, speaker 141, user interface 124) on a weekly basis.

Upon performing the self-test, control system 106 may provide the results to a user via one or more of speaker 141, user interface 124, and/or transmitting of results to a remote server or device. In some embodiments, the results of the self-test indicate the one or more components have failed and thus are not functioning properly. Upon indication of a failure of the self-test, control system 106 may cause one or more of speaker 141 or user interface 124 to provide the failing results to a user to make them aware that one or more components have failed the self-test and that user intervention may be required. In some embodiments, control system 106 is configured to store the results of the self-test indicating that one or more components have failed the self-test and may provide them to a user upon request.

Control system 106 may communicate with the one or more failing components to

determine the issue causing the failure of the self-test. For example, upon indication that blower 104 is not functioning properly, control system 106 may communicate with one or more pressure sensors of blower 104 to determine whether there is an occlusion or may determine whether blower 104 is drawing any current indicating a short, cut wire, or detached wire. In some embodiments, upon indication of a failure, control system 106 may cause speaker 141 to output an audio indication and/or cause user interface 124 to output a visual indication. In some embodiments, control system 106 causes writing device 113 to write to transmitting device 117 or a remote device the results of the self-test.

In some embodiments, speaker 141 is configured to output audio (e.g., an alert or alarm) when a failure is detected during use or a self-test of one or more components. Speaker 141 may be coupled to control system 106 and control system 106 may be configured to transmit a signal to speaker 141 to an output a noise or audio output. Control system 106 may be configured to perform a test on speaker 141. In some embodiments, control system 106 is configured to send a signal to speaker 141 to output audio and measure the amount of current drawn by speaker 141 in response to the signal. Control system 106 may compare the measured amount of current drawn to a baseline current reading when speaker 141 is operating correctly. Control system 106 may detect a failure of speaker 141 if the measured amount of current drawn deviates from the baseline current reading.

In some embodiments, control system 106 is configured to measure the impedance of current drawn by speaker 141 to determine if speaker 141 is receiving the correct amount of current to function correctly. Failure of speaker 141 may result in medical device unit 102 being unable to provide an audio alert or alarm in response to a failure of one or more components during use or a self-test. Upon failure of speaker 141, control system 106 may transmit a signal to a backup audio source, such as a buzzer that is configured to provide an audio alert. In some embodiments, the buzzer is a piezoelectric buzzer. However, the buzzer may be any type of buzzer or device capable of generating an audible alarm or alert. In some embodiments, control system 106 is configured to detect failure of speaker 141 during a self-test or during in real-time, such as during use of medical device unit 102.

In some embodiments, medical device unit 102 of system 100 is configured to administer

a status check, store the results of the status check, and then power down. In some embodiments, medical device unit 102 is configured to perform a self-test while medical device unit 102 is in storage or otherwise not in active use (e.g., in a powered down state). The results of the status check may be stored on memory 115, which may be configured to transmit the results without receiving power from medical device unit 102. For example, medical device unit 102 may power on, administer a status check, store the results of the status check on transmitting device 117 and/or memory 115, and then power down. Transmitting device 117 may be configured to transmit the results only when interrogated by an external source. The external source may be a receiving or reading device that provides power to transmitting device 117 enabling transmitting device 117 to transmit the results. This allows medical device unit 102 to conserve power as it does not need to power on to transmit the results of the status check and enables medical device unit 102 to provide results at any time upon interrogation by a user. However, transmitting device 117 may be configured to automatically transmit results on a periodic or aperiodic basis. For example, transmitting device 117 may automatically transmit results without user intervention.

In some embodiments, medical device unit 102 includes one or more pressure sensors.

For example, medical device unit 102 may include one or more pressure sensors coupled to blower 104. Medical device unit 102 may perform a self-test on blower 104 using the one or more pressure sensors. In some embodiments, controls system 106 sends a signal to blower 104 and receives a pressure reading from the pressure sensor coupled to blower 104. The pressure sensor may be disposed proximate the outlet of blower 104 to measure the pressure reading of air outputted by blower 104. Control system 106 may transmit an alert or warning based on the pressure reading of the pressure sensor coupled to blower 104 being too low. For example, control system 106 may transmit an alert or alarm if the pressure generated by blower 104 does not reach a predetermined threshold level for a given pulse width modulation (PWM) of blower 104. In some embodiments, the predetermined threshold level ranges from approximately 5 cm H2O to approximately 40 cm H2O at a PWM of approximately 25 to approximately 90. For example, the predetermined threshold level may be approximately 5 cm H2O at a PWM of approximately 25 or approximately 40 cm H2O at a PWM of approximately 90.

In some embodiments, blower 104 includes a tachometer to measure the RPMs of blower 104. Control system 106 may transmit an alert or warning based on the RPM measurement of the tachometer of blower 104 being too low. For example, control system 106 may transmit an alert or alarm if the RPMs of blower 104 does not reach a predetermined RPM level for a given PWM of blower 104. In some embodiments, the predetermined RPM level is greater than 3,000 RPMs at a PWM of 25. However, predetermined RPM level may be greater than 500 RPMs, 1,000 RPMs, 2,500 RPMs, 5000, RPMs, or 10,000 RPMs at a PWM ranging from 25 to 100.

In some embodiments, control system 106 is configured to transmit a signal to blower 104 and measure the current draw of blower 104 based on the signal. For example, control system 106 may send a signal to blower 104 to rotate at a specific RPM and may determine if the current being drawn by blower 104 to rotate at the specific RPM deviates from a normal or standard amount of current that blower 104 should draw to rotate at the specific RPM. In some embodiments, the RPM of blower 104 is dependent on the current drawn by blower 104 from, for example, power supply 108. Control system 106 may be configured to measure the current drawn by blower 104 for a specific RPM and determine if blower 104 is functioning improperly. In some embodiments, control system 106 activates blower 104 for a predetermined amount of time and measures the current and waveform during the predetermined amount of time and compares the current and waveform to a baseline current and baseline waveform when blower 104 is operating. For example, control system 106 may include data associated with how much baseline current is required to drive blower 104 for the predetermined amount of time and may compare the measured current of blower 104 to the baseline current. The predetermined amount of time may be less than 1 second, less than 2 seconds, less than three seconds, less than four seconds, less than five seconds, less than ten seconds, or greater than ten seconds. In some embodiments, the predetermined amount of time is any amount of time sufficient to determine how much current is being drawn by blower 104.

In some embodiments, blower 104 has a run time of less than a second. For example, during a self-test or status check of blower 104, blower 104 may run for less than a second to determine the functionality of blower 104 (e.g., pressure, RPMs, oxygen leakage). However, blower 104 may have a run time of less than 2 seconds, 3 seconds, 5 seconds, 10 seconds, 30 seconds, 1 minute, 5 minutes, 10 minutes, or greater than 10 minutes. In some embodiments, blower 104 is configured to be able to run for long periods of time without degradation in pressure or RPMs. For example, blower 104 may be configured to have a run time of greater than 30 minutes, 60 minutes, 1 day, 1 week, 1 month, 1 year, 3 years, or 5 years.

In some embodiments, medical device unit 102 includes an accelerometer. Control system 106 may be configured to receive measurements from the accelerometer and may request readings from the accelerometer during a self-test. The accelerometer may indicate whether medical device unit 102 is moving, has been moved, or has been dropped. In some embodiments, control system 106 determines if measurement from the accelerometer exceeds a threshold value indicating that medical device unit 102 has been dropped and may be damaged. The accelerometer may also indicate whether medical device unit 102 has been moved and an indication of movement based on the measurements from the accelerometer may be included in the status data. In some embodiments, the accelerometer provides orientation of the medical device unit 102. Medical device unit 102 may include a gyroscope to provide orientation information to control system 106.

Medical device unit 102 may include an optical sensor (e.g., infrared sensor, passive

infrared sensor). The optical sensor of medical device unit 102 may be coupled to control system 106, which may be coupled to user interface 124. Optical sensor may be configured to detect the amount of ambient light and decrease the brightness of user interface 124 based on the detected amount of ambient light to thereby decrease the power consumption of user interface 124. In some embodiments, optical sensor is configured to detect the presence of a user proximate to or viewing user interface 124. In the presence of a user, user interface 124 may display the status or status data of medical device unit 102. However, optical sensor may not detect a user or may detect the absence of a user proximate medical device unit 102 and may cause user interface 124 to not display the status or status data of medical device unit 102 since a user isn't present to view user interface 124. This allows for a reduction in power consumption since user interface 124 does not need to be illuminated unless the optical sensor detects the presence of a user viewing user interface 124.

In some embodiments, medical device unit 102 includes one or more environmental sensors. The environmental sensors may include a temperature sensor, a barometric sensor, a gas sensor, and a humidity sensor. Medical device unit 102 may include a temperature sensor to measure the ambient temperature and/or internal temperature of medical device unit 102. In use, ambient temperature may affect the performance of one or more components of medical device unit 102, such as blower 104, pressure sensors, speaker 141, battery, or any other component of medical device unit 102. In some embodiments, changes to the ambient temperature detected by the temperature sensor results in changes to parameters of the self-test. For example, the pressure thresholds associated with blower 104 may vary based on the ambient temperature detected by the temperature sensor. In yet another example, the performance of the battery or power supply 108 may vary based on the reading by temperature sensor resulting in threshold levels of alarms for low battery levels varying. In some embodiments, medical device unit 102 outputs an alarm if temperature reading of temperature sensor deviates from a temperature range. The temperature range may be the range of temperatures that medical device unit 102 can adequately perform in. The temperature range may be from −50° C. to 50° C.

Medical device unit 102 may also include one or more gas sensors. For example, medical device unit 102 may include a first oxygen sensor for measuring the oxygen levels within medical device unit 102 due to leakage and a second oxygen sensor for measuring the oxygen concentration of gas/air delivered to a patient coupled to medical device unit 102 via breathing circuit 200. The first oxygen sensor may be configured to transmit an alert when the oxygen levels within medical device unit exceed a predetermined threshold. For example, due to fire hazard concerns, medical device unit 102 may prevent the buildup of air/gas within medical device unit 102 by allowing a small amount of air/gas within medical device unit 102 to leak out. Medical device unit 102 may vent out the oxygen when first oxygen sensor detects that the oxygen level within medical device unit 102 is above a pre-determined amount. For example, medical device unit 102 may be configured to vent or leak out air/oxygen when the first oxygen sensor determines that there is more than 15%, 20%, 25%, 30%, 35%, 40%, 45%, or 50% oxygen within medical device unit 102. In some embodiments, medical device unit 102 is configured to continuously vent or leak out air/oxygen to keep the amount of oxygen within medical device unit 102 at or below 15%, 20%, 25%, 30%, 35%, 40%, 45%, or 50%.

In some embodiments, the first oxygen sensor is an ambient oxygen sensor, and the second oxygen sensor is a galvanic oxygen sensor. The first oxygen sensor may be used to calibrate the second oxygen sensor. For example, the ambient oxygen sensor may be a highly sensitive oxygen sensor and may be used to calibrate the galvanic oxygen sensor, which may degrade overtime and during use such that it requires re-calibration. However, the second oxygen sensor may be used to calibrate the first oxygen sensor. In some embodiments, a signal oxygen sensor is used to measure the oxygen levels within medical device unit 102 and for measuring oxygen concentration of gas/air delivered to the patient.

Medical device unit 102 may include an altitude sensor, such as an altimeter or barometric pressure sensor. In some embodiments, controls system 106 receives measurements from the altitude sensor and transmits corrections to other sensors, such as the oxygen sensors. For example, control system 106 may determine that medical device unit 102 is at a higher altitude and thus the oxygen levels are lower compared to sea level. Control system 106 may calibrate the oxygen sensors based on the altitude measurements from the altitude sensor. For example, control system 106 may apply a correction factor to the oxygen sensors based on the altitude measurements from the altitude sensor. Medical device unit 102 may also include humidity sensor to detect the levels of ambient humidity. Medical device unit 102 may output an alarm or alert if the humidity levels detected by the humidity sensor exceed a predetermined threshold. For example, excess moisture, such as that detected by the humidity sensor, may cause damage to one or more components of medical device unit 102 or may one or more components of medical device unit 102 to perform incorrectly (e.g., gas sensors, battery, speaker 141).

In some embodiments, during a self-test and/or upon startup, medical device unit 102 is configured to perform diagnostic readings of blower 104, power supply 108, writing device 113, memory 115, transmitting device 117, and/or control system 106. Medical device unit 102 may be configured to perform a self-test of one or more components of medical device unit 102 sequentially or simultaneously. In some embodiments, medical device unit 102 is configured to test blower 104 without coupling medical device unit 102 to breathing circuit 200. In some embodiments, medical device unit 102 is configured to receive software updates associated with the self-test. For example, medical device unit 102 may receive remote software updates changing predetermined time periods and/or threshold values. In some embodiments, medical device unit 102 includes indicator 134 to provide a status of system 100. Indicator 134 may be an indicator configured to provide the status of system 100 and/or medical device unit 102. For example, indicator 134 may indicate whether medical device unit 102 is damaged, inoperable, and/or functionally properly. Indicator 134 may be an LED and control system 106 may transmit a status to indicator 134 causing indicator 134 to illuminate a specific color and flash at specific frequency. However, indicator 134 may be a transmitter configured to transmit an outgoing signal. In some embodiments, indicator 134 is configured to continuously transmit an outgoing signal regarding the status of medical device unit 102. For example, indicator 134 may be configured to continuously transmit a signal without being requested to transmit a signal. Indicator 134 may transmit a signal indicating all components of medical device unit 102 are functioning correctly.

In some embodiments, indicator 134 continuously transmits a signal until an error occurs, which interrupts the signal transmission resulting in indicator 134 no longer transmitting a signal. For example, indicator 134 may illuminate a specific color (e.g., green) and may indicate to a user that an error occurs when the specific color is no longer being illuminated or a different color is being illuminated by indicator 134. A user may use a receiver to determine whether indicator 134 is transmitting a signal and whether an error has occurred based on the transmission ceasing. In other embodiments, indicator 134 is configured to transmit a first signal when medical device unit 102 is functioning correctly without significant errors and is configured to transmit a second signal when an error occurs. The first signal may be different than the second signal. Indicator 134 may transmit a signal wirelessly via radio frequency, WiFi, cellular signal, Bluetooth, near field communication, or any other type of wireless modality.

In some embodiments, indicator 134 provides a status of medical device unit 102 without

requiring a user to interact with or power on medical device unit 102. For example, indicator 134 may be coupled to a power supply separate from power supply 108 and may be configured to illuminate to provide an indication of a status to a user without the user interacting with medical device unit 102. Indicator 134 may be coupled to control system 106, which is configured to transmit a signal to indicator 134 to illuminate. In some embodiments, when control system 106 transmits a signal to indicator 134, control system 106 simultaneously transmits a signal to an external receiving device to indicate the status of medical device unit 102.

In some embodiments, control system 106 transmits a signal to indicator 134 and/or an external receiving device regardless of whether the external receiving device is proximate to medical device unit 102 or whether the external receiving device is requesting data from control system 106. For example, control system 106 may be configured to transmit a signal regardless of whether a device is listening or whether a device is requesting a signal from control system 106. In some embodiments, control system 106 is configured to always be transmitting a signal to indicator 134 when medical device unit 102 is functioning correctly or operating normally. For example, when medical device unit 102, indicator 134 may always be illuminated (e.g., green).

In some embodiments, indicator 134 is configured to display or flash different colors of light based on the status of medical device unit 102. For example, indicator 134 may display or flash the color green when medical device unit 102 is operating normally, display or flash the color red when medical device unit 102 is malfunctioning, or display or flash the color yellow/orange when medical device unit 102 has an error, but can still function. Indicator 134 may flash a color or have a constant illumination. Indicator 134 may be any color desired and may alternate between different colors depending on the status of medical device unit 102. In some embodiments, indicator 134 is coupled to a beacon power supply to ensure that indicator 134 is able to continuously provide an indication for the status of medical device unit 102. The beacon power supply may be different than power supply 108.

Control system 106 may perform a self-test or status check without user intervention and may cause indicator 134 to illuminate based on the results of the self-test or status check. A user may view medical device unit 102 after the self-test or status check has been performed and may view indicator 134. Upon viewing indicator 134, a user may be able to determine the status of medical device unit 102 and if there are any errors associated with medical device unit 102 without having to interact with medical device unit 102. Interacting with medical device unit 102 may including actuating one or more buttons on medical device unit 102, powering on medical device unit 102, or engaging with user interface 124.

In practice, a user may view indicator 134 immediately after the self-test or status check has been performed or may view indicator 134 after a duration of time as elapsed since the self-test or status check has been performed. In some embodiments, control system 106 is configured to transmit a signal to indicator 134 regardless of the power status of medical device unit 102. In other words, indicator 134 may be configured to always receive a signal from control system 106 regardless of the power status of medical device unit 102. This may be due to control system 106 and indicator 134 each having their own power supply separate from power supply 108 or control system 106 and indicator 134 sharing a power supply separate from power supply 108. In some embodiments, indicator 134 has a low power sensor configured to receive a signal from control system 106 to illuminate based on the status of a performed self-test or status check.

In some embodiments, housing 132 also includes indicator 133. Indicator 133 may be similar to indicator 134. Indicator 133 may also indicate the status of medical device unit 102 and may be used to provide alerts to the user regarding an alarm condition. For example, indicator 133 being green may indicate normal operation of medical device unit 102. However, indicator 133 flashing amber, red, yellow, or orange may indicate a malfunction or error with medical device unit 102. In some embodiments, the degree of flashing of indicator 133 indicates the severity of the error. Indicator 134 may also indicate the battery status associated with power supply 108. For example, indicator 134 being green may indicate that the battery of medical device unit 102 is fully charged. Indicator 134 being other colors, such as red, orange, yellow, amber, and/or flashing may indicate a malfunction or power level of the battery.

In some embodiments, control system 106 is configured to perform a status check on a battery coupled to medical device unit 102. For example, control system 106 may be configured to determine whether a new battery inserted into medical device unit 102 is defective or damaged. During use of medical device unit 102, replacing of the battery does not reset medical device unit 102. For example, upon removing an old battery and inserting a new battery, medical device unit 102 may remember settings that were used when the old battery was inserted into medical device unit 102. This allows for continued use of medical device unit 102 without having medical device unit 102 reset when a new battery is inserted. In some embodiments, control system 106 consistently and/or routinely stores setting information of medical device unit 102 into memory 115 such that when medical device unit 102 loses power, such as replacing of the battery, upon startup medical device unit 102 continues operating with the most recent settings. In some embodiments, control system 106 is configured to determine the age of a battery inserted into medical device unit 102. For example, when a battery is inserted into medical device unit 102, control system 106 may perform a self-test or status check to determine the age of the battery.

In some embodiments, medical device unit 102 includes an internal clock. The internal clock may be configured to measure the duration of time that medical device unit 102 loses power (e.g., replacing the battery). Upon loss of power during replacing the battery, medical device unit 102 may default to the most recent used settings if the loss of power duration is under a predetermined time. For example, if medical device unit 102 is without power for under 5 minutes, then medical device unit 102 may default to the most recent used settings upon receiving power. However, the predetermined time may be under 1 minutes, under 2 minutes, under 3 minutes, under 4 minutes, under 6 minutes, under 7 minutes, under 8 minutes, under 10 minutes, under 15 minutes, or under 20 minutes. If medical device unit 102 is without power for greater than the predetermined time, then medical device unit 102 may reset back to default settings when medical device unit 102 receives power again. In some embodiments, medical device unit 102 is a ventilator such that upon losing power for under the predetermined time, upon receiving power again, medical device unit 102 continues providing ventilation at the same settings used prior to medical device unit 102 losing power.

Control system 106 may receive status data regarding the functional status of system 100 and store the status data with memory 115. Status data may be any information describing the functionality and operation of any component of system 100. For example, the status data may be the results of a self-test or status check performed by medical device unit 102. Writing device 113 may access the status data stored within memory 115 to write the status data to transmitting device 117 stored within medical device unit 102. A user may wirelessly access the status data from transmitting device 117 without requiring medical device unit 102 to power on. This allows medical device unit 102 to be able to transmit status data wirelessly and without powering on, thereby reducing power consumption. For example, a user may interrogate transmitting device 117 and receive the status data while medical device unit 102 is powered off. A user may interrogate transmitting device 117 using a receiving or reading device.

In some embodiments, the receiving or reading device may be configured to provide power to transmitting device 117, allowing it to transmit the status data without requiring medical device unit 102 to power on. In some embodiments, medical device unit 102 does not provide power to transmitting device 117. In some embodiments, transmitting device 117 may transmit status data without requiring a user to physically contact medical device unit 102. For example, transmitting device 117 may only transmit status data upon request from an external source, such as a reading or receiving device. However, transmitting device 117 may be configured to autonomously and automatically transmit status data on a periodic or aperiodic basis. In some embodiments, status data may include one or more of the following a serial number, a software version, accessory information, power supply information, date of last status data request, date of last operation, date of manufacture, date of last repair, replaced components, results of prior status checks, usage reports, accessory information, battery information, and battery status.

Control system 106 may automatically test medical device unit 102 on a periodic, scheduled, aperiodic basis, or random basis. For example, control system 106 may power on medical device unit 102 and test all the components of medical device unit 102 on a periodic basis such as, for example, every month, every 3 months, or every 6 months. Control system 106 may automatically perform a self-test on medical device unit 102 on a random basis such as varying amounts of time between self-tests. Control system 106 may perform self-tests on a random basis to ensure that self-tests are performed on medical device unit 102 when the internal clock is damaged, defective, or miscalibrated. In some embodiments, a user may schedule specific dates for control system 106 to power on medical device unit 102 and test one or more components of medical device unit 102. For example, medical device unit 102 may be configured to have a programmable schedule to pre-schedule self-tests of one or more components. Pre-scheduled self-tests may be used when large quantities of medical device units 102 are being transported for use such that defective, damaged, or inoperable medical device units 102 are identified prior to use. In another embodiment, control system 106 may power on medical device unit 102 and test all the components of medical device unit 102 on an aperiodic basis. For example, tests may need to run more frequently the longer medical device unit 102 is in storage. In some embodiments, control system 106 tests medical device unit 102 without user intervention. Control system 106 may perform a self-test upon indication that medical device unit 102 has been moved based on measurements from the accelerometer and/or based on changes detected by one or more environmental sensors (e.g., temperature sensor, humidity sensor, altitude sensor).

In some embodiments, control system 106 is configured to autonomously power on medical device unit 102 to perform a self-test or status check. For example, control system 106 may be configured to periodically or aperiodically wake medical device unit 102 to perform a self-test or status check. In some embodiments, control system 106 performs a self-test or status check in a “silent mode” such that a user is unable to notice that a self-test or status check is being performed. For example, control system 106 may perform a self-test or status check without turning on user interface 124, speaker 141, indicator 133, or indicator 134 and without user intervention. In some embodiments, a user requests control system 106 to perform a self-test or status check by interacting with user interface 124 or engaging with/actuating button 126. For example, a user may actuate button 126 for approximately 5 seconds to initiate a self-test or status check on medical device unit 102. However, a user may actuate button 126 for 3 seconds, 4, seconds, 6 seconds, or greater than 6 seconds to initiate a self-test or status check. In some embodiments, a user transmits a request to control system 106 to perform a self-test on one or more components of medical device unit 102. Upon completion of the self-test, control system 106 may transmit the status data (e.g., results of the self-test) to the user, store the status data, display the status data, illuminate indicator 133 or indicator 134, and/or provide an audio indication of the status data via speaker 141.

In some embodiments, control system 106 causes activation of indicators 133, indicator 134, user interface 124, or speaker 141 to indicate that medical device unit 102 is performing a self-test or status check. For example, user interface 124 may illuminate with a message indicating that a self-test or status check is being performed or speaker 141 may produce an audio output to indicate to the user that a self-test or status check is being performed. In some embodiments, user interface 124 is configured to display the results of the self-test or status check. User interface 124 may display the results, status data, and/or other information associated with the self-test. In some embodiments, user interface 124 displays prior self-test results and may allow a user to view self-test results from a specific time period. In some embodiments, upon powering on, medical device unit 102 displays to a user results of a self-test completed upon powering on of medical device unit 102.

In practice, medical device unit 102 may start-up/boot-up and perform a self-test. The self-test may include testing of sensors (e.g., pressure sensors, oxygen sensors, temperature sensors, accelerometer, tachometer, gyroscope). In some embodiments, the self-test includes measuring the current drawn from one or more components (user interface 124, blower 104, speaker 141, control system 106) to determine whether the one or more components are receiving adequate current. The measurement of the current drawn by the one or more components may also determine if there is a short, cut wire, or detached wire.

In some embodiments, upon detection of an error during a self-test or status check during start-up, medical device unit 102 may reboot and run another self-test to determine if the error is still present. If so, medical device unit 102 may transmit an alert or alarm via user interface 124 and/or speaker 141. In some embodiments, during use, medical device unit 102 receives an error and in response to the error may reboot. During rebooting of medical device unit 102, medical device unit 102 may perform a self-test or status check to determine if the error is still present. If the error is still present, control system 106 may determine whether the error is a critical error affecting the ability of medical device unit 102 to provide treatment to a patient. If the error is a critical error, medical device unit 102 may alert the user to cease using the current medical device unit 102. However, if the error is not a critical error, medical device unit 102 may provide an option to the user to ignore the error and continue using medical device unit 102 without receiving continuous alerts and alarms.

In some embodiments, medical device unit 102 includes a secondary control system coupled to control system 106. The secondary control system may monitor control system 106 to determine whether there are any errors associated with the functioning of control system 106. Upon rebooting of medical device system 102, the second control system may also reboot and continue monitoring control system 106. Upon the second control system detecting an error in control system 106, the second control system may provide the user an alert or alarm. In some embodiments, medical device unit 102 includes a reserve clock (e.g., watchdog clock) coupled to control system 106. The reserve clock may be configured to monitor the duration of time it takes to reboot medical device unit 102. In some embodiments, the reserve clock is the same as the internal clock. In use, if the reboot of medical device unit 102 exceeds a predetermined time period, then control system 106 may alert the user. The predetermined time period may be approximately 1 minute, approximately 2 minutes, approximately 3 minutes, approximately 5 minutes, or approximately 10 minutes.

In some embodiments, the state of medical device unit 102 after powering on is dependent on the prior state of medical device unit 102. For example, medical device unit 102 may receive an error while in use and may power down and restart. Upon restarting, medical device unit 102 may resume in the state and same settings that medical device unit 102 was in prior to the restarting. If medical device unit 102 is powered down for greater than a predetermined amount of time, medical device unit 102 may restart into a new state with reset default settings and not the prior state and settings that medical device unit 102 was in prior to the restart. In some embodiments, if the battery is replaced in a time less than the predetermined amount of time during restarting of medical device unit 102, medical device unit 102 restarts in the same state and settings that medical device unit 102 was in prior to restarting. If the battery is replaced in a time a greater than the predetermined amount of time, then medical device unit 102 may restart in a new state with reset default settings and not in the same state and settings that medical device unit 102 was in prior to restarting. In some embodiments, if the battery is replaced in a time greater than the predetermined amount of time, control system 106 is configured to perform a self-test or status check on the newly replaced battery. The predetermined amount of time may be approximately 1minute, approximately 2 minutes, approximately 3 minutes, approximately 5 minutes, or approximately 10 minutes.

In some embodiments, the rebooting of medical device unit 102 takes less than 1 minute to ensure that medical device unit 102 can quickly resume providing treatment to a patient. For example, rebooting of medical device unit 102 takes less than 50 seconds, 40 seconds, 30 seconds, 20 seconds, 15 seconds, 10 seconds, or 5 seconds. In some embodiments, rebooting of medical device unit 102 takes less than 3 minutes, less than 5 minutes, or less than 10 minutes. In some embodiments, control system 106 detects an error in breathing circuit 200 resulting in rebooting of medical device unit 102. If upon rebooting, medical device unit 102 continues to detect an error in breathing circuit 200, medical device unit 102 may require a user to swap out or replace breathing circuit 200 with another breathing circuit 200.

Control system 106 may be configured to store and generate a history log based on previously performed self-tests. In some embodiments, control system 106 is configured to store all status data from each self-test. Control system 106 may store each failure of a self-test and actions taken in response to the failure in the history log. Control system 106 may automatically transmit the history log on a periodic, aperiodic, or pre-scheduled basis. However, control system 106 may be configured to transmit the history log in response to a request.

In some embodiments, medical device unit 102 is configured to provide remediating instructions on fixing an error. For example, control system 106 may request a self-test or status check on one or more components and may receive an error. In response to the received error, control system 106 may cause user interface 124 and/or speaker 141 to provide instructions to a user. For example, if control system 106 receives an error from one or components, such as blower 104, user interface 124 may display graphics and/or a video instructing a user how to fix the error associated with blower 104 (e.g., adjusting the position of blower 104, removing occlusions from blower 104, replacing blower 104 and/or one or more components, installing a software update). In some embodiments, medical device unit 102 provides step-by-step instructions to a user via user interface 124 or a microphone such that the user progresses through the instructions at their own pace. For example, a user may interact with user interface 124 to progress through step-by-step instructions or may use their voice to instruct medical device unit 102 to skip to the next instructions or repeat instructions for a specific step.

In some embodiments, control system 106 detects an error while medical device unit 102 is in use. For example, an error may occur resulting in medical device unit 102 not functioning correctly while being used with a patient. Control system 106 may detect the error while medical device unit 102 is in use and may provide instructions in real-time to a user to correct the error to allow medical device unit 102 to return to functioning correctly. In some embodiments, control system 106 causes speaker 141 provide audio instructions to the user to fix an error. Speaker 141 may be configured to correspond to graphics and/or videos presented by user interface 124. In some embodiments, user interface 124 is configured to receive inputs from a user to advance or repeat instructions associated with fixing an error.

In some embodiments, control system 106 is configured to transmit text, audio, images, graphics, and/or video regarding fixing an error to an electronic device associated with the user. Control system 106 may be configured to provide instructions for fixing an error while medical device unit 102 is powered on. In practice, control system 106, which may be coupled to a power supply separate from power supply 108, may receive an error from one or more components while medical device unit 102 is powered off. Upon powering on medical device unit 102, control system 106 may cause user interface 124 and/or speaker 141 to provide instructions to a user to fix an error. However, control system 106 may provide instructions to user while medical device unit 102 is in use. In some embodiments, control system 106 receives an error while medical device unit 102 is in use and provides instructions in real time. Control system 106 may receive an error without performing a status check or self-test. For example, during use of medical device unit 102, an error may occur within breathing circuit 200 or patient interface 300. Control system 106 may detect that an error has occurred and may provide instructions to a user via user interface 124 and/or speaker 141 on how to address and correct the error in real-time. For example, user interface 124 may provide audio and/or video instructions to the user on how to replace a component, reposition a component, remove occlusions from a component, install a software update, or any other type of remediating instructions.

In some embodiments, control system 106 is configured to provide instructions to a user regarding the appropriate therapy or the optimal treatment for a patient. For example, user interface 124 may provide audio, video, graphics, and/or text to a user recommending services and treatments to provide to a patient. In use, various parameters (e.g., PIP, TV, RR, PEEP, I:E ratio, ventilation mode, flow rate) of medical device unit 102 may need to be adjusted by a user when medical device unit 102 is being used for treating a patient. Control system 106 may instruct user interface 124 to provide instructions to a user for adjusting these parameters. In some embodiments, control system 106 provides instructions to a user via user interface 124 based on the current treatment that medical device unit 102 is providing to a patient. For example, medical device unit 102 may be a ventilator providing oxygen to a patient at a specific tidal volume and user interface 124 may provide instructions to a user to vary the tidal volume based on information control system 106 receives from various sensors or components of medical device unit 102.

In practice, multiple medical device units 102 may be stockpiled or placed in storage for a long period of time prior to use and thus may require multiple tests to ensure that medical device unit 102 is functioning properly prior to use. In practice, previous medical devices require physical intervention (e.g., opening device, turning device on, etc.) to determine if the device is functioning properly. This is an inefficient use of resources and also depletes the devices power supply. In some embodiments, medical device unit 102 automatically powers on and runs tests of the various components stored within medical device unit 102 to generate status data. Control system 106 of medical device unit 102 may store the status data within memory 115. Writing device 113 may access the status data from memory 115. In some embodiments, control system 106 sends the status data directly to writing device 113. Writing device 113 may write the status data to transmitting device 117, such as an RFID tag, which stores the status data. After writing device 113 writes the status data to transmitting device 117, medical device unit 102 may shut off to conserve power. A user may retrieve the status data by using a receiving or reading device, such as an RFID reader, to wirelessly receive status data while medical device unit 102 is powered off.

In some embodiments, one transmitting device 117 may be associated with a plurality of medical device units 102. For example, a pallet or stockpile of medical device units 102 may be in communication with a single transmitting device 117 such that each writing device 113 of each medical device unit 102 writes the results of the self-test (e.g., status data) to the single transmitting device 117. This allows a user to determine whether medical device units 102 in a large volume of medical device units 102 are defective, damaged, or inoperable. This configuration allows for the monitoring and surveillance of a stockpile or large quantity of medical device units 102 without having to interact or be adjacent to each one. Further, this provides for a quick assessment of whether a stockpile or collection of medical device units 102 are ready for use.

In some embodiments, medical device unit 102 may only transmit the status data when requested by a user. For example, control system 106 may receive the status data and writing device 113 may receive the status data from control system 106 and/or memory 115. Writing device 113 may write the status data to transmitting device 117, which may store the status data. Transmitting device 117 may only transmit the status data when requested by a user, such as by a user placing a receiving device adjacent to medical device unit 102 or by the user interacting with medical device unit 102 via user interface 124 or buttons 126, 128. However, transmitting device 117 may be configured to autonomously transmit status data without user intervention. For example, transmitting device 117 may detect that a receiving device is located proximate to medical device unit 102 and may automatically and autonomously transmit status data. In some embodiments, transmitting device 117 is configured to autonomously and automatically transmit status data on a periodic or aperiodic basis to receiving device located proximate or remote to medical device unit 102.

In some embodiments, control system 106 receives information from accessories associated with medical device unit 102. For example, the accessories may each include one or more wireless transmitting devices, disposed within or on them, configured to transmit information to control system 106 when interrogated. Medical device unit 102 may include one or more accessories that each may store information to their respective wireless transmitting devices. The wireless transmitting devices associated with each of the accessories may store information about the accessory such as the product type, expiration, model number, serial number, modification, last test, last use, etc. In some embodiments, control system 106 may interrogate the wireless transmitting devices to receive accessory information about the accessories and store that accessory information within memory 115. Control system 106 may be configured to interrogate all accessories proximate to medical device unit 102. For example, control system 106 may interrogate all accessories in predetermined radius to ensure that medical device unit 102 has all the accessories it requires to function. Writing device 113 of medical device unit 102 may write the accessory information about the accessories to transmitting device 117, which may store the accessory information along with the status data. In some embodiments, control system 106 is configured to interrogate any accessory or device within a predetermined proximity and/or radius and write any information received to transmitting device 117 and/or memory 115.

In some embodiments, medical device unit 102 is configured to create a mesh network with surrounding medical device units, allowing for the transmitting and receiving of status data associated with multiple medical device units. Medical device unit 102 may be configured to interrogate one or more medical device units within close proximity or a predetermined radius. Each medical device unit may be configured to interrogate adjacent medical device units and store information relating to the status data of the medical device units in close proximity such that each medical device unit includes status data of all medical device units in a surrounding area. For example, medical device unit 102 may interrogate and write status data related to all medical device units in a surrounding area to transmitting device 117 and/or memory 115. This allows a user to only have to interrogate a single medical device unit to obtain information from all medical device units in a surrounding area. The surrounding area may be a radius of at least 1 foot, at least 2 feet, at least 3 feet, at least 4 feet, at least 5 feet, at least 10 feet, or at least 25 feet. In some embodiments, medical device unit 102 may interrogate surrounding medical device units to determine the status of accessories associated with the surrounding medical device units. This allows a user to determine which medical device units and/or accessories need attention by interrogating only medical device unit 102.

In some embodiments, the mesh network created by medical device unit 102 and the surrounding medical device units allows control system 106 to map the location of surrounding medical device units along with status data associated with each medical device unit. This allows for a user to interrogate medical device unit 102 and obtain status data for all surrounding medical device units, in addition to determining the location of the surrounding medical device units. Determining the location of the surrounding medical device units, allows a user to easily determine, based on the status data which medical device unit is not functioning properly and allows the user to easily find and replace the malfunctioning medical device unit. In some embodiments, medical device unit 102 is configured to transmit a map of the locations of the surrounding medical device units to the user when requested.

In some embodiments, medical device unit 102 may include a wireless network module, such as a WiFi chip/card, configured to communicate with control system 106 and one or more external devices. The one or more external devices may include writing device 113, transmitting device 117, a server, a computer, a mobile device, or an external transmitter. The wireless network module may receive a signal from an external device causing medical device unit 102 to power on and administer a status check. The status data resulting from the status check on start-up may be stored within memory 115 and/or may be wirelessly transmitted to writing device 113, which may be disposed outside of medical device unit 102. Writing device 113 may then write status data to transmitting device 117, which may be stored within, on, or external to housing 132 of medical device unit 102. In some embodiments, writing device 113 and transmitting device 117 are each disposed proximate to medical device unit 102. In alternative embodiments, writing device 113 and transmitting device 117 are each disposed remote to medical device unit 102.

In some embodiments, medical device unit 102 may provide status data via additional methods. In some embodiments, indicator 133 on medical device unit 102 may provide the status of medical device unit 102. For example, indicator 133 may be an LED indicator or status light that may display a green light if there are no malfunctions or may display a red light if there are malfunctions. When a red light is displayed, indicating a malfunction of medical device unit 102, a user may retrieve the status data from transmitting device 117 to receive detailed results of the test to address the malfunction. In some embodiments, a receiving device and/or writing device 113 is disposed adjacent to medical device unit 102 to constantly receive status data from or write data to transmitting device 117 whenever medical device unit 102 runs tests of the various components stored within medical device unit 102. For example, whenever a test of medical device unit 102 is run, control system 106 and/or memory 115 may provide transmitting device 117 with the status data. Transmitting device 117 may transmit the status data to the receiving device upon receipt of the status data. The receiving device may alert a user when there are any malfunctions contained within the status data or may transmit the status data to a central server or database for a user to access. This may allow for the monitoring of multiple medical device units 102 in storage without having a user having to periodically check on the status of each medical device unit 102 in storage. Further, this allows for real-time broadcasting of status data from medical device unit 102.

In some embodiments, medical device unit 102 may include speaker 141, additional lights, and/or additional display screens. Medical device unit 102 may be configured to alert a user via one or more of user interface 124, indicator 133, indicator 134, user interface 124, display screens, or other modes of alerting a user. For example, medical device unit 102 may provide an alert, warning, or message to a user by text, audio, or visual indicators. Medical device unit 102 may provide alerts for one or more of electricity supply failure, medical device unit 102 being switched off while in a specific mode, inspiratory and PEEP pressure exceeding predetermined threshold values, or inspiratory and PEEP pressure going below a do not achieve minimum threshold values, tidal volume or respiratory rate (RR) not being achieved or being exceeded, medical device unit 102 being disconnected from power, obstruction of blower 104, or apnea. As discussed herein, medical device unit 102 may be configured to provide instructions to a user on how to fix an error detected by control system 106 via one or more of user interface 124, speaker 141, or an electronic device associated with a user.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

What is claimed is:

1. A medical device comprising:
an electro-mechanical pneumatic system disposed within a housing of the medical device and coupled to one or more components; and
a control system coupled to the electro-mechanical pneumatic system, the control system configured to:
without receiving an external request, automatically transmit to one or more of the electro-mechanical pneumatic system and the one or more components a request for status data regarding a status of one or more of the electro-mechanical pneumatic system and the one or more components;
receive the status data from one or more of the electro-mechanical pneumatic system and the one or more components; and
output a signal based on the status data from one or more of the electro-mechanical pneumatic system and the one or more components.

2. The medical device of claim 1 further comprising:
a transmitting device configured to receive the status data from the control system.

3. The medical device of claim 2, wherein the transmitting device is coupled to a plurality of medical devices and is configured to receive a plurality of status data from the plurality of medical devices.

4. The medical device of claim 1, wherein the control system is configured to output the signal to one or more of a speaker, a user interface, a storage device, an external device, a beacon, a writing device, a transmitting device, and an indicator.

5. The medical device of claim 1 further comprising:
a wake-up controller coupled to the control system, the wake-up controller configured to power on the medical device prior to control system transmitting a request for the status data.

6. The medical device of claim 1, wherein the control system is further configured to:
display on a user interface the status data via a display screen or a light indicator.

7. The medical device of claim 1, wherein the control system is further configured to:
measure a current drawn from the electro-mechanical pneumatic system; and
based on the current drawn from the electro-mechanical pneumatic system, determine a status of the electro-mechanical pneumatic system.

8. The medical device of claim 1, wherein the control system is further configured to:
measure a current drawn from a speaker coupled to the control system; and
based on the current drawn from the speaker, determine a status of the speaker.

9. The medical device of claim 1, wherein the control system is further configured to:
receive an input to power on the medical device;
in response to the input, power on the medical device; and
administer a status check to one or more of the electro-mechanical pneumatic system and the one or more components to obtain the status data of one or more of the electro-mechanical pneumatic system and the one or more components.

10. The medical device of claim 1, wherein the control system is configured to communicate with one or more other medical device units in a surrounding area to receive status data associated with the one or more other medical device units.

11. The medical device of claim 1 further comprising:
one or more accessories, wherein the control system is configured to receive accessory information associated with the one or more accessories.

12. The medical device of claim 1, wherein the control system includes a low power controller configured to transmit the request for the status data.

13. The medical device of claim 1 further comprising:
a beacon configured to provide an indication representative of the status data.

14. The medical device of claim 1 further comprising:
a power supply disposed within the housing and coupled to the control system, the power supply configured to receive a charge based on the status data.

15. The medical device of claim 1, wherein the one or more components include one or more of a power supply, a sensor, a valve, a wake-up controller, a speaker, and a user interface.

16. The medical device of claim 1, wherein the control system is further configured to:
power on the medical device prior to transmitting the request for status data; and
power down the medical device upon outputting the signal based on the status data.

17. A medical device having a control system, the control system configured to:
without receiving an external request, automatically transmit to one or more components of the medical device a request for status data regarding a status of the one or more components;
receive the status data from the one or more components; and
output a signal based on the status data from the one or more components, wherein the signal is outputted to one or more of a speaker, a user interface, a storage device, an external device, a beacon, a writing device, a transmitting device, and an indicator.

18. The medical device of claim 14, wherein the control system is further configured to:
based on the status data, provide a charge to a power supply disposed within the medical device.

19. A medical device comprising:
an electro-mechanical pneumatic system having a blower, the electro-mechanical pneumatic system disposed within the medical device and coupled to one or more components; and
a controller coupled to the electro-mechanical pneumatic system and a control system, the controller configured to:
automatically power on the medical device; and
transmit a signal to the control system to initiate a status check of one or more of the electro-mechanical pneumatic system and the one or more components of the medical device.

20. The medical device of claim 14, wherein the controller automatically powers on the medical device without receiving an external request.
